(19) United States
(12) Patent Application Publication
(10) Pub. No.: US 2021/0282715 A1
(43) Pub. Date: Sep. 16, 2021

(54) Patch, Patch System And Method For Using A Patch

(71) Applicants: Bionime Corporation, Taichung City (TW); Bionime Corporation, Taichung City (TW)

(72) Inventor: Chun-Mu Huang, Taichung City (TW)

(21) Appl. No.: 17/200,190

(22) Filed: Mar. 12, 2021

(30) Foreign Application Priority Data
Mar. 13, 2020 (TW) 109108480

(51) Int. Cl. A61B 5/00; A61B 5/145

(52) U.S. Cl. CPC A61B 5/68335; A61B 5/14532

(57) ABSTRACT

A patch, comprising: a backing disposing thereon an adhesive layer having an adhesive surface for being adhered to a skin surface; and a peelable sheet detachably adhered to the adhesive surface for preserving the adhesive surface and providing a supporting force for the backing. The peelable sheet includes an inner peelable sheet and an outer peelable sheet. After the inner peelable sheet is peeled off, the outer peelable sheet continuously provides the supporting force to allow the backing to be adhered easily and evenly.

### CROSS REFERENCE TO RELATED APPLICATIONS

The application claims the benefit of Taiwan Patent Application No. 109108480, filed on Mar. 13, 2020, at the Taiwan Intellectual Property Office, the disclosures of which are incorporated herein in their entirety by reference.

### FIELD OF THE INVENTION

The present invention relates to a patch, a patch system and a method for using a patch. More particularly, the present invention relates to a patch, a patch system and a method for using a patch suitable for a physiological parameter detection device.

### BACKGROUND OF THE INVENTION

A patch-type physiological parameter detection device uses a base patch to adhere the physiological parameter detection device to a skin surface of a user. Considering the durability and comfort when adhering the physiological parameter detection device to the skin surface, and in order to prevent the base patch from falling off during use, a patch having a greater area is usually covered on the base patch to increase the strength of the base patch of the physiological parameter detection device (such as a transmitter) adhered to the skin.

For example, a traditional blood glucose management uses a blood glucose monitor system (BGMS). Under this system, in order to monitor the glucose concentration of interstitial fluids, a diabetic patient must take blood by needling the skin several times a day for measurement. The American Diabetes Association (ADA) recommends that it is best to measure the glucose concentration three times a day or more, but the patient will find it hard to endure the pain and inconvenience. Therefore, in order to alleviate the suffering of the patient, continuous glucose monitoring (CGM) was developed. Its detection principle is that a soft needle, that is, a sensor, is implanted under the skin to continuously test the glucose level in the interstitial fluid, and the retention period can be up to fourteen days, which greatly reduces the burden on the patient. The continuous blood glucose monitor is a patch-type physiological parameter detection device.

Considering the comfort, the patch used in the aforementioned patch-type physiological parameter detection device is generally made of a relatively soft material. However, this kind of material easily bunches up and gets wrinkled, which makes it difficult for a user to adhere it to the skin smoothly, especially when it is applied with one hand.

In view of this, it is necessary to develop a patch that easily and smoothly adheres to the skin.

### SUMMARY OF THE INVENTION

In accordance with one aspect of the present invention, a patch is disclosed. The patch includes a backing and a peelable sheet. The backing disposes thereon an adhesive layer having an adhesive surface for being adhered to a skin surface. The peelable sheet is detachably adhered to the adhesive surface for preserving the adhesive surface and providing a supporting force for the backing, and includes an inner peelable sheet and an outer peelable sheet, wherein both sheets are relatively easily peelable off from the adhesive surface, wherein after the inner peelable sheet is peeled off, the outer peelable sheet continuously provides the supporting force for the backing to allow the backing to be easily and evenly adhered.

In accordance with another aspect of the present invention, a patch system is disclosed. The patch system includes a physiological parameter detection device, a backing and a peelable sheet. The physiological parameter detection device has a base patch for being adhered to a skin surface via the base patch. The backing disposes thereon an adhesive layer having an adhesive surface, wherein the backing is adhered to the skin surface via the adhesive surface. The peelable sheet is detachably adhered to the adhesive surface, for preserving the adhesive surface and providing a supporting force for the backing, and including an inner peelable sheet and an outer peelable sheet, wherein the both sheets are relatively easily peelable off from the adhesive surface, wherein after the inner peelable sheet is peeled off, the outer peelable sheet continuously provides the supporting force for the backing.

In accordance with a further aspect of the present invention, a method for using a patch is disclosed, wherein the patch is cooperated with a physiological parameter detection device having a base patch to be adhered to a skin surface of a user. The method includes steps of: providing a patch including a backing and a peelable sheet, wherein the backing has a first patch area and a second patch area, the peelable sheet is attached to the first and the second patch areas for providing a supporting force to the backing, and the peelable sheet includes an inner peelable sheet and an outer peelable sheet, in which the both sheets are relatively easily peelable off; peeling off the inner peelable sheet to expose a surface of the first patch area corresponding to the inner peelable sheet; after the inner peelable sheet is peeled off, continuously providing the supporting force for the backing through the outer peelable sheet so as to allow the backing to be adhered easily and evenly; and peeling off the outer peelable sheet to expose a surface of the second patch area corresponding to the outer peelable sheet so as to increase an adhesive stability of the physiological parameter detection device on the skin surface.

### BRIEF DESCRIPTION OF THE DRAWINGS

FIG. 1 shows a rear view of a patch according to an embodiment of the present invention.

FIG. 2 shows a front view of the patch according to an embodiment of the present invention.

FIG. 3 shows a longitudinal section of the patch according to an embodiment of the present invention.

FIG. 4 shows a use state of the patch according to an embodiment of the present invention.

FIG. 5 shows a use state of the patch according to an embodiment of the present invention.

FIG. 6 shows a use state of the patch according to an embodiment of the present invention.

FIG. 7 shows a patch system according to an embodiment of the present invention.

FIG. 8 shows the patch system according to an embodiment of the present invention.

FIG. 9 shows the patch system according to an embodiment of the present invention.

FIG. 10 shows the patch system according to an embodiment of the present invention.

FIG. 11 shows the patch system according to an embodiment of the present invention.

FIG. 12 shows a patch according to an embodiment of the present invention.

FIG. 13 shows a patch according to an embodiment of the present invention.

FIG. 14 shows a patch according to an embodiment of the present invention.

FIG. 15 shows a patch according to an embodiment of the present invention.

FIG. 16 shows a patch according to an embodiment of the present invention.

### DETAILED DESCRIPTION OF THE PREFERRED EMBODIMENTS

The technical content, features and effects of the present invention will be clearly presented by the following detailed descriptions of preferred embodiments.

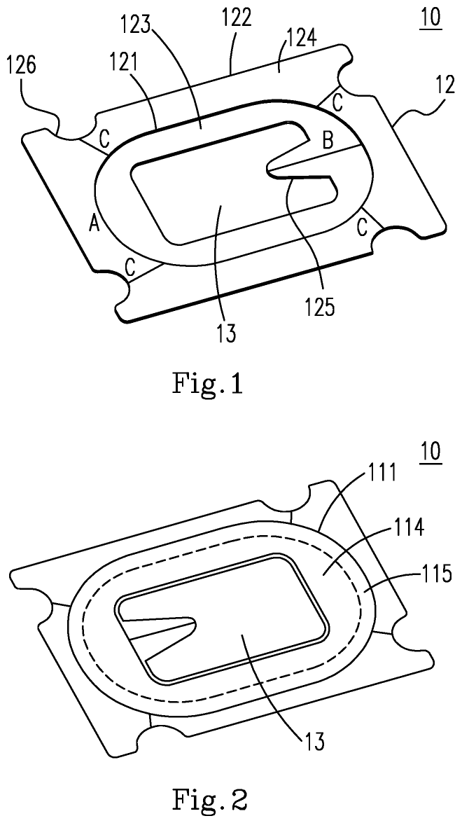

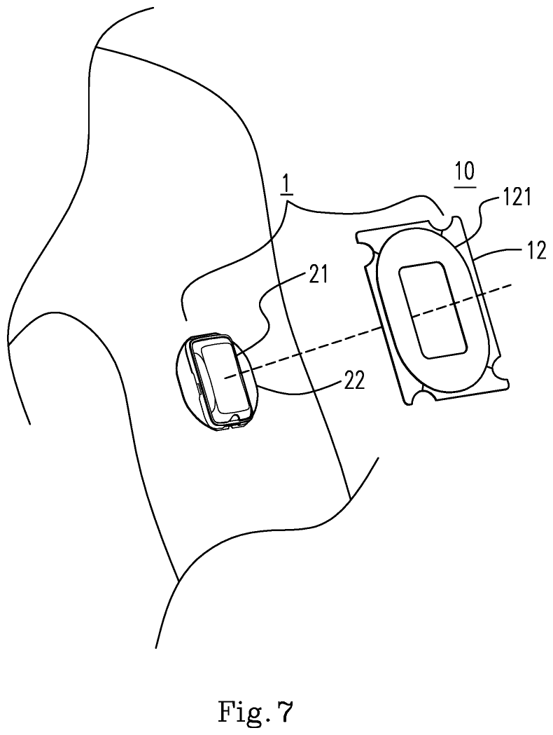

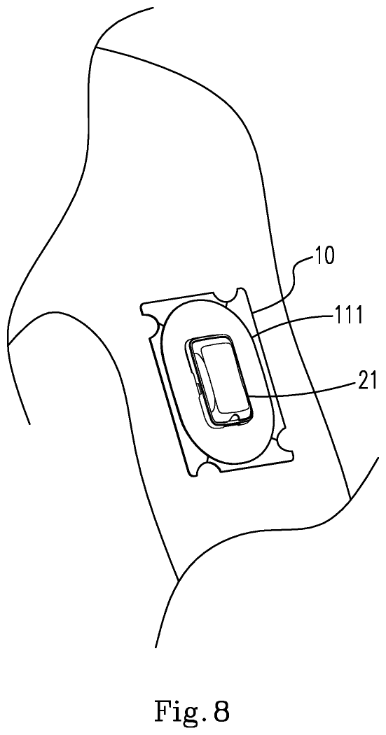

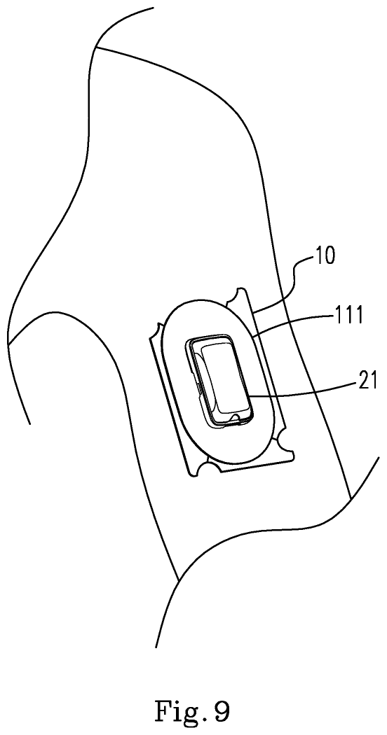

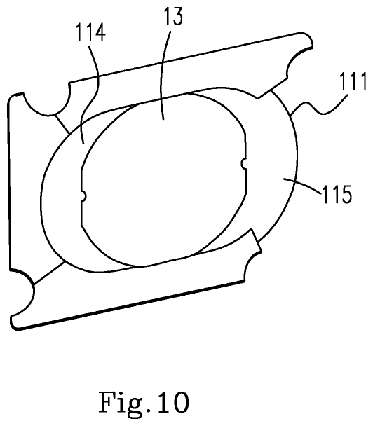

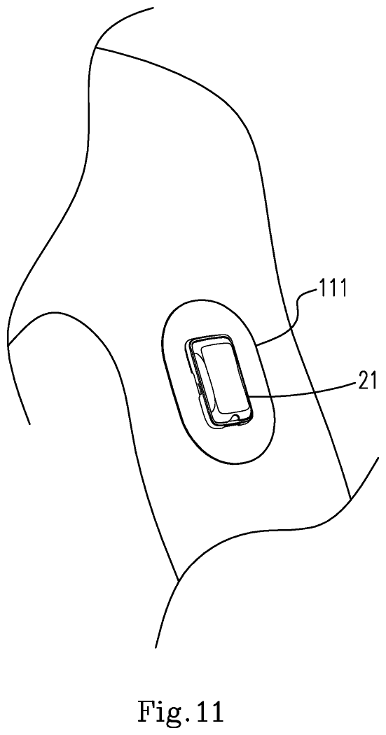

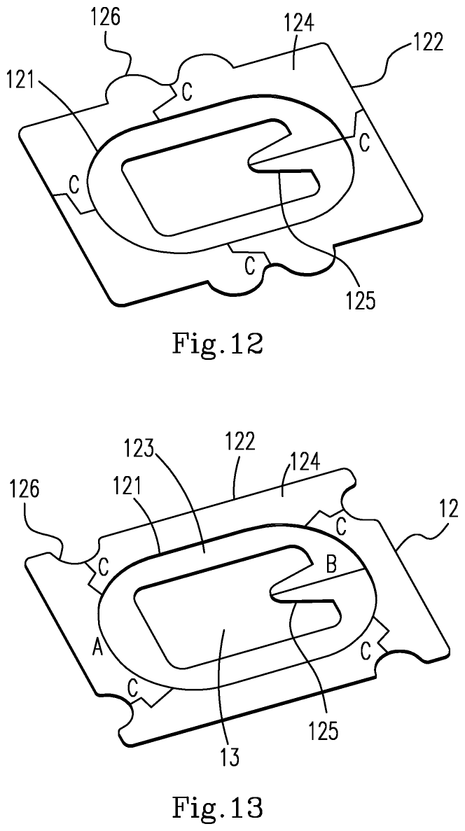

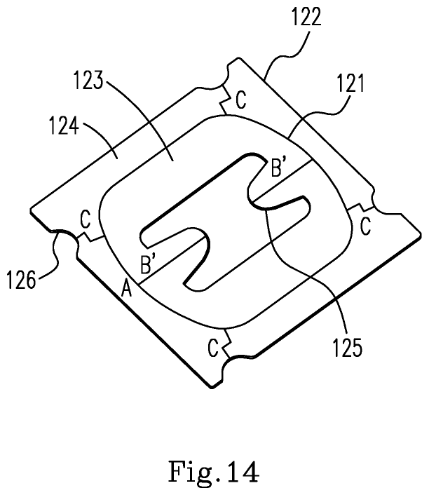

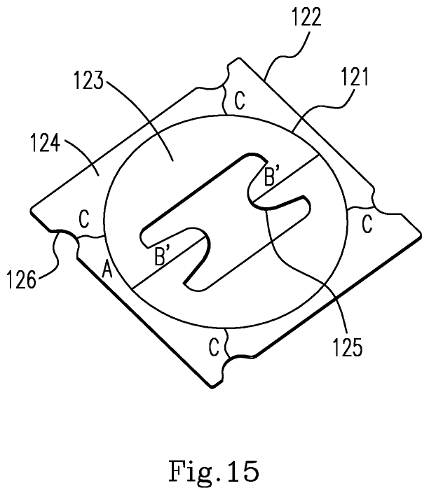

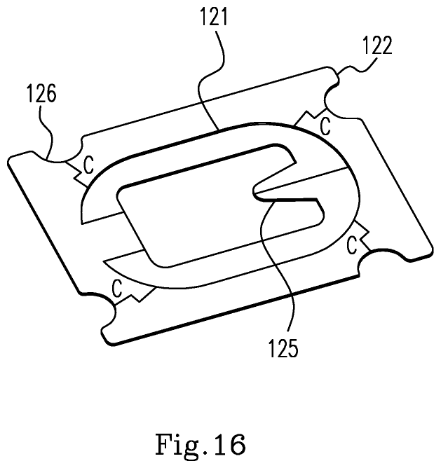

Please refer to FIGS. 1-16. The first embodiment of the present invention provides a patch system 1. First please refer to FIGS. 7-11, the patch system 1 includes a physiological parameter detection device 21; a backing 111; and a peelable sheet 12. The physiological parameter detection device 21 has a base patch 22, and the physiological parameter detection device 21 is adhered to a skin surface via the base patch 22. The physiological parameter detection device 21 may be a combination of a transmitter, a sensor, and a base in a continuous glucose monitoring system.

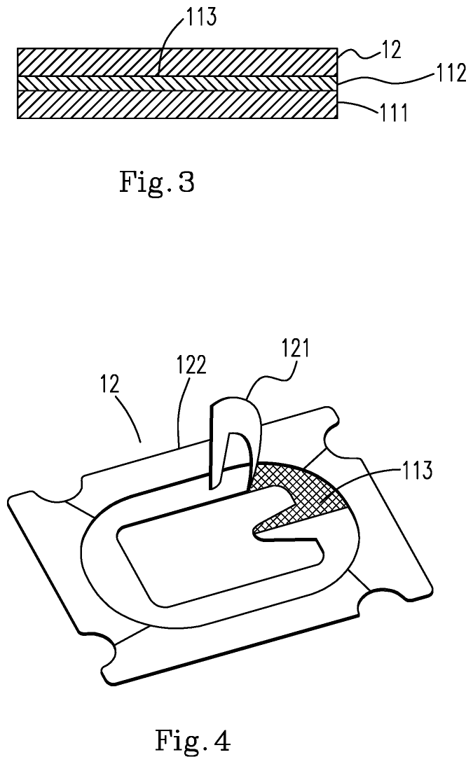

Please refer to FIG. 3. An adhesive layer 112 having an adhesive surface 113 is disposed on the backing 111. The backing 111 is adhered to the skin surface via the adhesive surface 113. The peelable sheet 12 is detachably adhered to the adhesive surface 113. The peelable sheet 12 is used to preserve the adhesive surface 113 of the backing 111 and provide a supporting (stretching-up) force to the backing 111.

Please refer to FIG. 1. The peelable sheet 12 includes an inner peelable sheet 121 and an outer peelable sheet 122, wherein the both sheets are relatively easily peelable off from the adhesive surface 113.

Please refer to FIGS. 5 and 7-8. After the inner peelable sheet 121 is peeled off, the outer peelable sheet 122 continuously provides the supporting force for the backing 111, and the backing 111 increases the strength of the physiological parameter detection device 21 attached to the skin surface.

Please refer to FIG. 1. The inner peelable sheet 121 and the outer peelable sheet 122 include at least one inner peelable sub-sheet 123 and at least one outer peelable sub-sheet 124, respectively.

Please refer to FIGS. 1 and 14-15. In the patch system 1, a first cutting line A may be disposed between the inner peelable sheet 121 and the outer peelable sheet 122. When the number of the inner peelable sub-sheet 123 is one, a second cutting line B may be disposed on the inner peelable sub-sheet 123. When the inner peelable sheet 121 includes a plurality of the inner peelable sub-sheets 123, a second cutting line B′ may be disposed between the inner peelable sub-sheets 123. At least one third cutting line C may be disposed between the outer peelable sub-sheets 124. The outer peelable sheet 122 may include, for example, four outer peelable sub-sheets 124.

Please refer to FIG. 2. In the patch system 1, the backing 111 have a shape of an ellipse. However, the backing may have a shape of a rectangle or a circle. The present disclosure is not limited thereto.

In the patch system 1, the backing 111 may have a flexibility. The backing 111 may be a paper, a cloth, or a plastic mesh, and the peelable sheet 12 may be a release paper or a release sheet.

Please refer to FIGS. 1-2 and 7. In the patch system 1, the backing 111 may have a region 13, a first patch area 114 and a second patch area 115. The first patch area 114 and the second patch area 115 are extended from the surrounding of the region 13 so as to encompass the region 13. The first patch area 114 and the second patch area 115 correspond to the inner peelable sheet 121 and the outer peelable sheet 122 respectively. The region 13 has an area greater than that of the physiological parameter detection device 21 and less than that of the base patch 22 of the physiological parameter detection device 21. The inner peelable sheet 121 has an inner shorter than either side of that of the first patch area 114 to protect the adhesive surface 113 of the backing 111.

The region 13 may have a geometric shape. The region 13 may be a hole having a geometric shape.

Please refer to FIGS. 1 and 12-16. In the patch system 1, the inner peelable sheet 121 and the outer peelable sheet 122 may be respectively provided with at least one inner auxiliary tearing part 125 and at least one outer auxiliary tearing part 126, in which the outer auxiliary tearing part 126 may be protrusive or recessive.

Please refer to FIGS. 1 and 16. In the patch system 1, the inner peelable sheet 121 may form a full circle or a non-full circle.

Please refer to FIGS. 1 and 12-16. The third cutting line C may be a curve, a polyline and a straight line. Compared with the straight line, when the third cutting line C is the curve or the polyline, the backing 111 can be kept flat and less likely to droop when the outer peelable sub-sheet 124 is peeled off.

Please refer to FIGS. 1-16. The second embodiment of the present invention provides a patch 10. The patch 10 includes a backing 111 and a peelable sheet 12. An adhesive layer 112 having an adhesive surface 113 is disposed on the backing 111. The backing 111 is adhered to a skin surface via the adhesive surface 113.

The peelable sheet 12 is detachably adhered to the adhesive surface 113. The peelable sheet 12 is used to preserve the adhesive surface 113 of the backing 111 and provide a supporting force to the backing 111. The peelable sheet 12 includes an inner peelable sheet 121 and an outer peelable sheet 122, wherein the both sheets are relatively easily peelable off from the adhesive surface 113. After the inner peelable sheet 121 is peeled off, the outer peelable sheet 122 continuously provides the supporting force for the backing 111 so as to perform a complete attaching of the backing 111 and allow the backing 111 to be adhered easily and evenly.

The backing 111 may have a region 13, a first patch area 114 and a second patch area 115. The first patch area 114 and the second patch area 115 are extended from the surrounding of the region 13. The first patch area 114 and the second patch area 115 correspond to the inner peelable sheet 121 and the outer peelable sheet 122 respectively. The region 13 has an area greater than that of a physiological parameter detection device 21 and less than that of a base patch 22 of the physiological parameter detection device 21.

The backing 111 and the peelable sheet 12 in the second embodiment of the present invention have the features as in the first embodiment of the present invention, and will not be described here.

Please refer to FIGS. 1-16. The third embodiment of the present invention provides a method for using the patch 10. The method includes the following steps.

Please refer to FIGS. 1-2. The patch 10 including the backing 111 and the peelable sheet 12 is provided. The backing 111 has the first patch area 114 and the second patch area 115. The peelable sheet 12 is attached to the first patch area 114 and the second patch area 115 for providing the supporting force to the backing 111, and includes an inner peelable sheet 121 and an outer peelable sheet 122, in which the both sheets are relatively easily peelable off. The inner peelable sheet 121 and the outer peelable sheet 122 include at least one inner peelable sub-sheet 123 and at least one outer peelable sub-sheet 124, respectively.

Please refer to FIG. 7. The physiological parameter detection device 21 having the base patch 22 is provided. The physiological parameter detection device 21 is adhered to the skin surface of the user via the base patch 22.

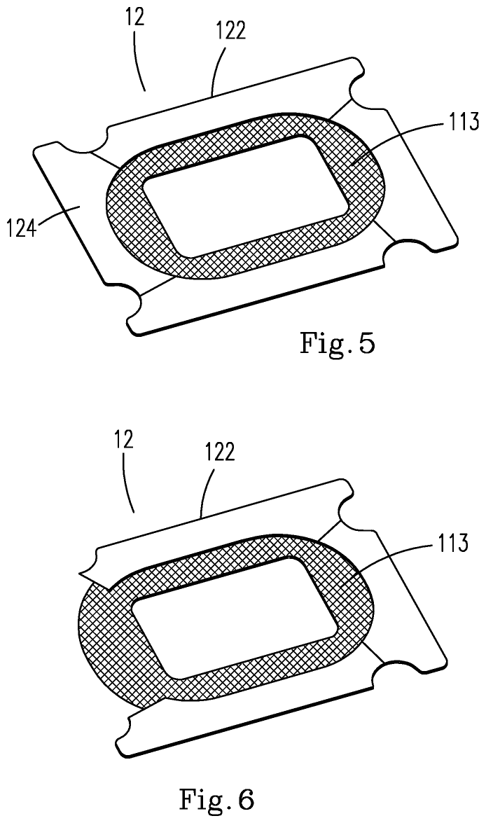

Please refer to FIGS. 4-5. The inner peelable sheet 121 is peeled off to expose a surface of the first patch area 114 corresponding to the inner peelable sheet 121.

Please refer to FIGS. 5 and 7-8. After the inner peelable sheet 121 is peeled off, the outer peelable sheet 122 continuously provides the supporting force for the backing 111 so as to perform a complete attaching of the backing 111 and allow the backing 111 to be adhered easily and evenly.

Please refer to FIGS. 6 and 9-11. The outer peelable sheet 122 is peeled off to expose a surface of the second patch area 115 corresponding to the outer peelable sheet 122. Thereby, the adhesive strength of the physiological parameter detection device 21 on the skin surface is enhanced through the backing 111.

### EMBODIMENTS

1. A patch includes a backing and a peelable sheet; the backing disposes thereon an adhesive layer having an adhesive surface for being adhered to a skin surface; and the peelable sheet is detachably adhered to the adhesive surface for preserving the adhesive surface and providing a supporting force for the backing, and including an inner peelable sheet and an outer peelable sheet, wherein the both sheets are relatively easily peelable off from the adhesive surface, wherein after the inner peelable sheet is peeled off, the outer peelable sheet continuously provides the supporting force for the backing to allow the backing to be easily and evenly adhered.
2. The patch according to Embodiment 1, wherein the inner peelable sheet and the outer peelable sheet include at least one inner peelable sub-sheet and at least one outer peelable sub-sheet, respectively.
3. The patch according to Embodiments 1-2, wherein: the inner peelable sheet and the outer peelable sheet include a plurality of inner peelable sub-sheets and a plurality of outer peelable sub-sheets, respectively; and a first cutting line is disposed between the inner peelable sheet and the outer peelable sheet, at least one second cutting line is optionally disposed between the inner peelable sub-sheets, and at least one third cutting line is optionally disposed between the outer peelable sub-sheets.
4. The patch according to Embodiments 1-3, wherein: the backing has a flexibility and is capable of being a paper, a cloth, or a plastic mesh, and wherein the peelable sheet is a release paper or a release sheet.
5. The patch according to Embodiments 1-5, wherein the backing has a region, a first patch area and a second patch area, in which the first and the second patch areas are extended from the surrounding of the region, and correspond to the inner peelable sheet and the outer peelable sheet respectively.
6. The patch according to Embodiments 1-6, wherein: the patch is able to be cooperated with a physiological parameter detection device having a base patch; and the region has an area greater than that of the physiological parameter detection device, but less than that of the base patch.
7. The patch according to claim 5, wherein the inner peelable sheet has an inner edge shorter than either side of that of the first patch area to protect the adhesive surface of the backing.
8. The patch according to Embodiments 1-7, wherein the region has a geometric shape.
9. The patch according to Embodiments 1-8, wherein the region is a hole having a geometric shape.
10. The patch according to Embodiments 1-9, wherein: the inner peelable sheet and the outer peelable sheet are respectively provided with at least one inner auxiliary tearing part and at least one outer auxiliary tearing part, in which the outer auxiliary tearing part is protrusive or recessive; and the inner peelable sheet forms a full circle or a non-full circle.
11. The patch according to Embodiments 1-10, wherein the third cutting line is a curve, a polyline or a straight line.
12. A patch system includes a physiological parameter detection device, a backing and a peelable sheet; the physiological parameter detection device has a base patch for being adhered to a skin surface via the base patch; the backing disposes thereon an adhesive layer having an adhesive surface, wherein the backing is adhered to the skin surface via the adhesive surface; and the peelable sheet is detachably adhered to the adhesive surface, for preserving the adhesive surface and providing a supporting force for the backing, and includes an inner peelable sheet and an outer peelable sheet, wherein the both sheets are relatively easily peelable off from the adhesive surface, wherein after the inner peelable sheet is peeled off, the outer peelable sheet continuously provides the supporting force for the backing.
13. The patch system according to Embodiment 12, wherein the inner peelable sheet and the outer peelable sheet include at least one inner peelable sub-sheet and at least one outer peelable sub-sheet, respectively.
14. The patch system according to Embodiments 12-13, wherein: the inner peelable sheet and the outer peelable sheet include a plurality of inner peelable sub-sheets and a plurality of outer peelable sub-sheets, respectively; and a first cutting line is disposed between the inner peelable sheet and the outer peelable sheet, at least one second cutting line is optionally disposed between the inner peelable sub-sheets, and at least one third cutting line is optionally disposed between the outer peelable sub-sheets.
15. The patch system according to Embodiments 12-14, wherein: the backing has a region, a first patch area and a second patch area, in which the first and the second patch areas are extended from the surrounding of the region, and correspond to the inner peelable sheet and the outer peelable sheet respectively.
16. The patch system according to Embodiments 12-15, wherein: the region has an area greater than that of the physiological parameter detection device and less than that of the base patch of the physiological parameter detection device.
17. The patch system according to Embodiments 12-16, wherein the region is a hole having a geometric shape.
18. The patch system according to Embodiments 12-17, wherein: the inner peelable sheet and the outer peelable sheet are respectively provided with at least one inner auxiliary tearing part and at least one outer auxiliary tearing part, in which the outer auxiliary tearing part is protrusive or recessive.
19. A method for using a patch, in which the patch is cooperated with a physiological parameter detection device having a base patch to be adhered to a skin surface of a user; the method includes steps of: providing a patch including a backing and a peelable sheet, wherein the backing has a first patch area and a second patch area, the peelable sheet is attached to the first and the second patch areas for providing a supporting force to the backing, and the peelable sheet includes an inner peelable sheet and an outer peelable sheet, in which the both sheets are relatively easily peelable off; peeling off the inner peelable sheet to expose a surface of the first patch area corresponding to the inner peelable sheet; after the inner peelable sheet is peeled off, continuously providing the supporting force for the backing through the outer peelable sheet so as to so as to allow the backing to be adhered easily and evenly; and peeling off the outer peelable sheet to expose a surface of the second patch area corresponding to the outer peelable sheet so as to increase an adhesive stability of the physiological parameter detection device on the skin surface.
20. The method according to Embodiments 19, wherein the inner peelable sheet and the outer peelable sheet include at least one inner peelable sub-sheet and at least one outer peelable sub-sheet, respectively.

What is claimed is:

1. A patch, comprising:
a backing disposing thereon an adhesive layer having an adhesive surface for being adhered to a skin surface; and
a peelable sheet detachably adhered to the adhesive surface for preserving the adhesive surface and providing a supporting force for the backing, and including an inner peelable sheet and an outer peelable sheet, wherein the both sheets are relatively easily peelable off from the adhesive surface,
wherein after the inner peelable sheet is peeled off, the outer peelable sheet continuously provides the supporting force for the backing to allow the backing to be easily and evenly adhered.

2. The patch according to claim 1, wherein the inner peelable sheet and the outer peelable sheet include at least one inner peelable sub-sheet and at least one outer peelable sub-sheet, respectively.

3. The patch according to claim 2, wherein:
the inner peelable sheet and the outer peelable sheet include a plurality of inner peelable sub-sheets and a plurality of outer peelable sub-sheets, respectively; and
a first cutting line is disposed between the inner peelable sheet and the outer peelable sheet, at least one second cutting line is optionally disposed between the inner peelable sub-sheets, and at least one third cutting line is optionally disposed between the outer peelable sub-sheets.

4. The patch according to claim 1, wherein:
the backing has a flexibility and is capable of being a paper, a cloth, or a plastic mesh, and wherein the peelable sheet is a release paper or a release sheet.

5. The patch according to claim 1, wherein the backing has:
a region;
a first patch area; and
a second patch area, in which the first and the second patch areas are extended from the surrounding of the region, and correspond to the inner peelable sheet and the outer peelable sheet respectively.

6. The patch according to claim 5, wherein:
the patch is able to be cooperated with a physiological parameter detection device having a base patch; and
the region has an area greater than that of the physiological parameter detection device, but less than that of the base patch.

7. The patch according to claim 5, wherein the inner peelable sheet has an inner edge shorter than either side of that of the first patch area to protect the adhesive surface of the backing.

8. The patch according to claim 5, wherein the region has a geometric shape.

9. The patch according to claim 5, wherein the region is a hole having a geometric shape.

10. The patch according to claim 1, wherein:
the inner peelable sheet and the outer peelable sheet are respectively provided with at least one inner auxiliary tearing part and at least one outer auxiliary tearing part, in which the outer auxiliary tearing part is protrusive or recessive; and
the inner peelable sheet forms a full circle or a non-full circle.

11. The patch according to claim 3, wherein the third cutting line is a curve, a polyline or a straight line.

12. A patch system, comprising:
a physiological parameter detection device having a base patch for being adhered to a skin surface via the base patch;
a backing, disposing thereon an adhesive layer having an adhesive surface, wherein the backing is adhered to the skin surface via the adhesive surface; and
a peelable sheet detachably adhered to the adhesive surface, for preserving the adhesive surface and providing a supporting force for the backing, and including an inner peelable sheet and an outer peelable sheet, wherein the both sheets are relatively easily peelable off from the adhesive surface,
wherein after the inner peelable sheet is peeled off, the outer peelable sheet continuously provides the supporting force for the backing.

13. The patch system according to claim 12, wherein the inner peelable sheet and the outer peelable sheet include at least one inner peelable sub-sheet and at least one outer peelable sub-sheet, respectively.

14. The patch system according to claim 13, wherein:
the inner peelable sheet and the outer peelable sheet include a plurality of inner peelable sub-sheets and a plurality of outer peelable sub-sheets, respectively; and
a first cutting line is disposed between the inner peelable sheet and the outer peelable sheet, at least one second cutting line is optionally disposed between the inner peelable sub-sheets, and at least one third cutting line is optionally disposed between the outer peelable sub-sheets.

15. The patch system according to claim 12, wherein the backing has:
a region;
a first patch area; and
a second patch area, in which the first and the second patch areas are extended from the surrounding of the region, and correspond to the inner peelable sheet and the outer peelable sheet respectively.

16. The patch system according to claim 15, wherein:
the region has an area greater than that of the physiological parameter detection device and less than that of the base patch of the physiological parameter detection device.

17. The patch system according to claim 15, wherein the region is a hole having a geometric shape.

18. The patch system according to claim 12, wherein:
the inner peelable sheet and the outer peelable sheet are respectively provided with at least one inner auxiliary tearing part and at least one outer auxiliary tearing part, in which the outer auxiliary tearing part is protrusive or recessive.

19. A method for using a patch, in which the patch is cooperated with a physiological parameter detection device having a base patch to be adhered to a skin surface of a user, comprising steps of:
providing a patch including a backing and a peelable sheet, wherein the backing has a first patch area and a second patch area, the peelable sheet is attached to the first and the second patch areas for providing a supporting force to the backing, and the peelable sheet includes an inner peelable sheet and an outer peelable sheet, in which the both sheets are relatively easily peelable off;
peeling off the inner peelable sheet to expose a surface of the first patch area corresponding to the inner peelable sheet;
after the inner peelable sheet is peeled off, continuously providing the supporting force for the backing through the outer peelable sheet so as to allow the backing to be adhered easily and evenly; and
peeling off the outer peelable sheet to expose a surface of the second patch area corresponding to the outer peelable sheet so as to increase an adhesive stability of the physiological parameter detection device on the skin surface.

20. The method according to claim 19, wherein the inner peelable sheet and the outer peelable sheet include at least one inner peelable sub-sheet and at least one outer peelable sub-sheet, respectively.
